(19) United States
(12) Patent Application Publication
(10) Pub. No.: US 2010/0010092 A1
(43) Pub. Date: Jan. 14, 2010

(54) USE OF MODAFINIL TO TREAT RESTLESS LEG SYNDROME

(75) Inventors: Thomas N. Lavin, Watchung, NJ (US); Catherine H. Koo, Watchung, NJ (US)

Correspondence Address: DIAMOND LAW OFFICE LLC, 1605 JOHN STREET, SUITE 102, FORT LEE, NJ, 07024, US

(73) Assignee: Arless Ltd.

(21) Appl. No.: 12/518,870

(22) PCT Filed: Dec. 19, 2007
(86) PCT No.: PCT/US2007/088211

§ 371 Date: Jun. 11, 2009

Related U.S. Application Data
(60) Provisional application No. 60/870,801, filed on Dec. 19, 2006.

(51) Int. Cl. A61K 31/165; A61P 43/00

(52) U.S. Cl. 514/618

(57) ABSTRACT

A method for relieving, treating, improving or attenuating one or more symptoms of RLS and related movement disorders such as PLMS, and snoring ICSD 780.53-1 is disclosed. The method includes the administration to a host afflicted with RLS or related disorder a pharmaceutically effective amount of a modafinil compound or a related compound. The method of the present invention is to treat a host to reduce or diminish snoring, the unpleasant leg sensations associated with unwanted leg movements and to diminish or eliminate the unwanted, involuntary leg movements at rest, awake or asleep, typically occurring in the evenings and at night.

### BACKGROUND OF THE INVENTION

1. Technical Field

The present invention relates to a treatment for Restless Leg Syndrome and related disorders.

2. Background Art

Restless Leg Syndrome (RLS) ICSD 780.52-5 and related disorders such as periodic limb movement in sleep (PLMS) ICSD classification 780.52-4, which is also called periodic limb movement disorder (PLMD) is estimated to afflict 2.5 to 10% of the worldwide population (Garcia-Burreguero, et al.), and is therefore, probably the most common movement disorder. However, RLS has not been widely recognized by the medical profession as clinically significant until recently. The poor recognition of RLS is probably due to the absence of symptoms during most of the day and the rather unusual and bizarre description of symptoms that occur almost exclusively at night.

RLS is characterized by an unpleasant sensation at rest that has been variously described as crawling, creeping, cramping, pulling or tightening and sometimes painful sensation in the legs that cause an almost irresistible urge to move the legs that is relieved by moving the legs. Oftentimes, the sensations culminate in the involuntary movement of the foot, leg or thigh. The movement provides only temporary relief. The phenomenon occurs typically at rest, late in the evening. RLS and related disorders are believed to be a common cause of insomnia (Fox). Although a percentage of RLS has an underlying condition such as iron deficiency, uremia, diabetes mellitus, diabetes mellitus, rheumatoid arthritis, and polyneuropathy, and can occur during pregnancy (O'Keefe), a large percentage of RLS is idiopathic.

Periodic limb movements in sleep (PLMS) are rhythmic extensions of the toes, dorsiflexion of the foot and ankles, or flexion of knee and hip that occur in a series of four or more consecutive movements (Montplaisir). Between 80 to 90% of RLS patients exhibit PLMS. Although PLMS has not been positively associated with waking up during sleep and insomnia, PLMS and RLS have definite adverse effects on the patient's quality of life, and PLMS can result in the patient awakening from sleep (Saletu 2002). Continuously irresistible urges to move while at rest during the evening is disconcerting at best and often violent jerkings during sleep disturb both the patient and sleep partners. Patients with RLS and PLMS sometimes have other associated sleep disorders such as snoring. A patient with RLS and PLMD can have many hundreds of sudden leg movements per night.

RLS is believed to be a chronic condition that may worsen in a significant percentage of the patients. Current drug treatment of RLS includes oral administration of dopamine agonists, benzodiazepines, narcotics, clonidine, gabepentin (O'Keefe, Joy, Wefter, et al., Trenkwaider, et al. Silber, et al. Saletu, et al.) and even magnesium (Hornyak, 1998) with L-dopa and dopamine agonists as first-line treatments. Dopamine agonists can cause major side effects including insomnia, dizziness and postural hypotension. Treatment with L-dopa itself results in about 80% of the patients treated with levodopa exhibiting augmentation of the symptoms that can occur within months of initiation of therapy (Allen). Patients taking the dopamine agonist Requip (Ropinirole), the only FDA approved drug for treatment of moderate to severe RLS, have reported additional complications such as constipation and daytime sleepiness. Among the earliest tests of efficacy of ropinirole for RLS used the decrease in Periodic Limb Movements in sleep (PLM in sleep) as the efficacy measure for success of the drug, (Saletu, M. et al. 2000). Ropinirole is not universally effective and approximately 30 to 50 percent of patients on Requip do not obtain relief (ropinirole package insert, US and International study results). Benzodiazepines and narcotics are habit forming and thus, undesirable. In addition, benzodiazepines can cause daytime drowsiness and confusion, unsteadiness and falls, and aggravation of sleep apnea (Silber). There are no known prescribed treatments for Primary snoring ICSD 780.53-1, which occurs without sleep apnea or hypoxia and is a different syndrome than that of sleep apnea ICSD 780.53-0. Therefore, there is a need for the availability of alternative treatment for individuals with RLS, PLMS, snoring and related disorders.

Modafinil (sold as Provigil™ prescription only) is presently sold in tablet form as a wakefulness-promoting agent for oral administration. Modafinil is a racemic compound. The chemical name for Modafinil is 2-[(diphenylmethyl)sulfiny]acetamide. The molecular formula is C₁₅H₁₅NO₂S and the molecular weight is 273.36. The precise mechanism or mechanisms through which promotes wakefulness is unknown. Modafinil has wake promoting actions similar to sympathomimetic agents including amphetamine and methylphenidate, although the pharmacologic profile is not identical to that of sympathomimetic amines. In addition to its wakefulness-effects and increased locomotor activity in animals, in humans, Provigil™ produces psychoactive and euphoric effects, alterations in mood, perception, thinking, and feelings typical of other CNS stimulants.

Conventional wisdom is that the optical enantiomers of modafinil have similar pharmacological actions in animals. However, the enantiomers of modafinil have different pharmacokinetics with the half-life of the I-isomer is approximately three times that of the d-isomer in humans. The enantiomers do not interconvert. Thus, the trough concentration (Cₘᵢₙₛₛ) of circulating modafinil after once daily dosing consists of 90% of the I-isomer in 10% of the d-isomer. The generally prescribed dose of Provigil™ is 200 mg given once a day. Doses up to 400 mg per day, given as a single dose, have been well tolerated, but there is no consistent evidence that this dose confers additional benefit beyond that of the 200 mg dose. Provigil™ tablets are supplied as 100 mg or 200 mg tablets.

In view of the above conventional wisdom in pharmacology as set forth in the Physican's Desk Reference® published by Thompson, it would be totally unexpected that modafinil and related compounds would have any sort of physical calming effect, and specifically any positive effect on individuals with RLS, PLMS, snoring, and related disorders.

### SUMMARY OF THE INVENTION

According to the present invention, a method for relieving, treating, improving or attenuating one or more symptoms of RLS and related movement disorders such as PLMS, and snoring ICSD 780.53-1 is disclosed. The method includes the administration to a host afflicted with RLS or related disorder or primary snoring a pharmaceutically effective amount of a modafinil compound or a related compound. The method of the present invention is to treat a host to reduce or diminish snoring, the unpleasant leg sensations associated with unwanted leg movements and to diminish or eliminate the unwanted, involuntary leg movements at rest, awake or asleep, typically occurring in the evenings and at night.

### DESCRIPTION OF THE DRAWINGS

Other advantages of the present invention are readily appreciated as the same becomes better understood by reference to the following detailed description when considered in connection with the accompanying drawings wherein:

FIG. 1 depicts data from Example 6.

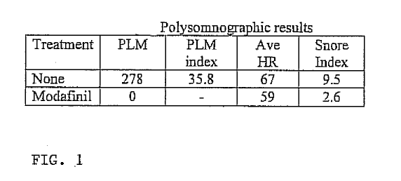

### DETAILED DESCRIPTION OF THE INVENTION

Modafinil or benzhydrylsulphinylacetamide is a drug, which works in the CNS as described in U.S. Pat. Nos. 4,177,290; 5,180,745; 5,612,379 and has been developed as a treatment to improve wakefulness in patients with excessive sleepiness associated with narcolepsy, obstructive sleep apnealhypopnea syndrome, and shift work sleep disorder. It is administered at a dose of 200 my daily as discussed above. Modafinil is DL-2-[diphenylmethyl)sulfinyl]acetamide (Provigil™) J Chromatogr B Biomed Sci Appl. 1999, which is racemic. Both D and L modafinil are described in U.S. Pat. No. 4,177,290 and preparation of the levorotary isomer is described in U.S. Pat. No. 4,927,855, and methods for the preparation of enantiomeric sulfinylacetamides are described in U.S. Patent Publication No. 2006/0086667, all of which are incorporated herein. The two optical enantiomers have similar pharmacologic action in mammals (FDA approved labeling for DNA 20-717/S-005 & S-008, 2004). When taken orally, the I-isomer predominates in the circulation. At steady state, total exposure to the I-isomer is approximately three times that for the d-isomer. The trough concentration (Cₘᵢₙₛₛ) of circulating modafinil after once daily dosing of racemic modafinil consists of 90% of the I-isomer and 10% of the d-isomer. Therefore, oral dosage of a racemic modafinil compound results predominantly in the circulation of the I-isomer.

Nuvigil, armodafinil, the R entantiomer according to the Cahn-Ingol-Prelog rules, r-2-((diphenylmethyl)sulfinyl)-acetamide or (−)benzhydrilsulfinylacetamide (U.S. Publication No. 2006/0086667), the I-isomer, as is approved for human use for the treatment of excessive sleepiness associated with narcolepsy, obstructive sleep apnea/hypopnea syndrome (OSA/HS) and shift work sleep disorder (SWSD). This is the same indication as racemic modafinil. Therefore, the drug is expected to perform as does racemic modafinil for the use in RLS, PLMS and snoring. Polymorphic forms of modafinil are described in U.S. Patent Publication No. 200610252835 and are incorporated herein. Modafinil's precise mechanism of action is unknown, but it is not a direct- or indirect-acting dopamine receptor agonist (FDA approved labeling, 2004 for NDA 20-717/S-005 & S-008), and therefore, it is an entirely different class of drugs from that of Requip, which is a dopaminergic agonist primarily used for Parkinson's syndrome. U.S. Pat. Nos. 4,098,824, 4,066,686, 4,127,722 and 4,177,290 describe families of wakefulness promoting benzhydrylsulphinyl derivatives and are incorporated herein.

As disclosed herein, and as used in the compositions and methods of the present invention, a modafinil compound may include any of the d or I or +or − enantiomers or isomers of modafinil such as Nuvigil or Armodafinil, or any of their polymorphic forms, a racemic mixture, and may be in an acid form, such as a metabolic acid of modafinil or a benzhydrylsulfinylacetic acid, a sulfone form, a hydroxylated form, a conjugated form such as a modafinil compound conjugated to a protein, a polysaccharide, a glucuronide or a sulfate, or a polymorphic form, it may include compounds containing isosteric replacements of the phenyl groups of modafinil, and polymorphic species or analogs of modafinil, or derivatives of cogeners and prodrugs, particularly those preparations that stimulate activity in the tuberomammillary nucleus (TMN) when administered to a mammal. In preferred embodiments, the modafinil compound is modafinil. Prodrugs are known in the art as compounds that are converted to the active agent (modafinil) in the body of a subject. Such compounds are described in U.S. Pat. Nos. 7,132,570; 4,177,290; 5,180,745; 5,612,379; 4,927,855 and U.S. Patent Publication Nos. 2006/0086667 and 2006/0252835 and are incorporated herein.

In a preferred embodiment, the compound is formulated as a tablet for oral administration. The modafinil compound may be formulated into a pharmaceutical composition using a form of the active ingredient described above. The formulation of modafinil containing tablets is, and such tablets may contain various inert ingredients typically used to prepare tablets, including but not limited to, lactose, corn starch, magnesium silicate, croscarmellose sodium, providone, magnesium stearate, or talc in any combination thereof.

Paradoxically, the present invention shows that dosages of the wakefulness promoting drug modafinil allows restful sleep and decreases or eliminates undesired involuntary leg movements at rest either awake or during sleep (PLMD/PLMS) in hosts afflicted with RLS with PLMS, the unpleasant sensation or urgency to move the leg and associated episodes or snoring.

In the method of the present invention, the compounds of the present invention can be administered in various ways, preferably orally by tablet. It should be noted that the preferred compounds can be administered as the compound or as a pharmaceutically acceptable salt and can be administered alone or as an active ingredient in combination with pharmaceutically acceptable carriers, diluents, adjuvants, and other vehicles. Although the compounds can be administered by various routes, such as intra or subcutaneously, or intramuscularly, preferably, the compounds are taken orally as a tablet. The Patient being treated is preferably a warm blooded animal and particular, mammals including man. The pharmaceutically acceptable carriers, diluents, adjuvants, and vehicles as well as other modes of administration generally refer to inert, non-toxic solids or liquid fillers, diluents, or incapsulating material not reacting with the active ingredients of the present invention.

The dosages of the present invention are preferably single doses taken prior to the patient retiring to sleep.

When administering the compound of the present invention, it is generally formulated in a unit dosage oral form, as a tablet. Alternative forms can be pharmaceutical formulation suitable for injection, including sterile aqueous solutions or dispersions and sterile powders for reconstitution into sterile injectable solutions or dispersions. The carrier can be a solvent or a dispersing medium containing, for example, water, ethanol, polyol (for example, glycerol, propylene glycol, liquid polytheylene glycol, and the like), suitable mixtures thereof, and vegetable oils.

Sterile injectable solutions can be prepared by incorporating the compounds utilized in practicing the present invention in the required amount of the appropriate solvent with various of the other ingredients, as described.

With specific regard to Modafinil, either the racemate or the D and/or L isomers can be administered. Also, the two optical enantiomers can be administered. Preferably, 10 to 100 mg of Modafinil are administered, and most preferably, 25 to 100 mg are administered as a pre-retiring to sleep dose.

The following examples demonstrate the efficacy of the present invention and unexpected results of administering a drug that would otherwise be expected to induce wakefulness, and yet provides significant relief to patients suffering from Restless Leg Syndrome and related disorders.

The invention is further described in detail by reference to the following experimental examples. These examples are provided for the purpose of illustration only, and are not intended to be limiting unless otherwise specified.

Thus, the invention should in no way be construed as being limited to the following examples, but rather, should be construed to encompass any and all variations which become evident as a result of the teaching provided herein.

### EXAMPLES

### Example 1

### Patient Administered Requip

An otherwise healthy non-obese 51 year old female with sleep study diagnosed PLMD and RLS and Primary snoring without apnea, 61 kg, took Requip, ropinirole HCL from 0.25 to 0.75 mg orally per day for PLMS and RLS for a period of over 8-12 months. PLM were severe enough to occasionally awaken her from sleep. The drug did not relieve symptoms and late evening leg movements and sensations prior to sleep continued. PLMS continued according to the partner. Multiple attempts at gaining relief were made over a period of 8-12 months. These attempts failed.

### Example 2

### Subject Administered 50 mg of Modafinil

A 53 year old non-obese female, 61 kg, with sleep study diagnosed PLMD with RLS without apnea, and Primary snoring, took modafinil 50 mg by mouth between 8 am and noon. No urge to move legs in the evenings occurred and no leg jerks during sleep were observed by her partner. The patient noted increased restfulness and her partner noticed an absence of limb movements and snoring while asleep.

### Example 3

### Subject Administered 100 mg of Modafinil

A 53 year old non-obese female, 61 kg, with sleep study diagnosed PLMD with RLS without apnea and Primary snoring, took modafinil 100 mg by mouth in the morning. No urge to move legs in the evenings occurred and no leg jerks during sleep were observed by her partner. The patient noted increased restfulness and her partner noticed an absence of limb movements and snoring while asleep.

### Example 4

### Continuous Use Evening Dosage

A 54 year old non-obese female, 61 kg, with sleep study diagnosed PLMD with RLS without apnea and Primary snoring, took modafinil 50 mg by mouth 1-3 hours prior to bedtime. Limb movements and the urge to move the limb and associated sensations while awake were greatly reduced after approximately 90 minutes. The patient then took 25 mg of modafinil as needed and noted that symptoms were either completely relieved or greatly diminished. Chronic use every day for periods of 5 to 7 days produced periods free from RLS and PLMD symptoms, which returned immediately after stopping the drug.

### Example 5

### Continuous Use Morning Dosage

A 53 year old non-obese female, 61 kg, with sleep study diagnosed PLMD with RLS with apnea and Primary snoring, took modafinil 50 mg or 25 mg by mouth in the morning. Limb movements and the urge to move the limb and associated sensations while awake were greatly reduced. The lower dosage of 25 mg failed to entirely remove symptoms. Either 50 my or 25 mg was used intermittently for a period of 14 months. Chronic use every day for periods of up to 11 days produced periods free from RLS and PLMD symptoms, which returned immediately after stopping the drug. The sleep partner noted a marked decrease in snoring.

### Example 6

### Sleep Study Results, Evening Dosage

A non-obese female subject with no outstanding medical history other than restless leg syndrome and PLMS of 6 years duration, and Primary snoring, age 54, 60 kg, was entered into an overnight sleep study in a specialist hospital sleep disorder center. Subject was monitored by polysomnographic recording method identifying sleep stages and their durations, detection and measurements of apneas, detection and measurement of PLM (periodic limb movements, snoring and measurement of pO₂, heart rate and arterial pressure. Sleep stages were identified using electroencephalographic and electromyographic recording. Oxygen saturation was measured by transcutaneous oximetric measurement and revealed no apnea. Baseline studies without drug administration showed that the subject exhibited multiple PLMs during sleep and significant snoring episodes. After oral administration of 50 mg modafinil in the evening PLMs were not detected during sleep as illustrated in Drawing 1. Snoring as represented by the Snore Index was reduced 70%.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

U.S. Patent Documents
U.S. Pat. No. 4,066,686
U.S. Pat. No. 4,098,824
U.S. Pat. No. 4,127,722
U.S. Pat. No. 4,177,290
U.S. Pat. No. 4,927,855
U.S. Pat. No. 5,180,745
U.S. Pat. No. 5,612,379
U.S. Pat. No. 6,346,548
U.S. Pat. No. 6,346,283
U.S. Pat. No. 6,348,500
U.S. Pat. No. 6,465,519
U.S. Pat. No. 6,492,396
U.S. Pat. No. 7,087,647
U.S. Pat. No. 7,132,551
U.S. Pat. No. 7,132,570
20060086667
20060252835

### OTHER REFERENCES

Montplaisir, J., Michaud, M., Petit, D., New Trends in Restless Leg Syndrome Research, Sleep Med. Rev., 10: 147-151, 2006
Hornyak, J., Feige, B., Riemann, D. and Voderholzer, U., Periodic leg movements in sleep and periodic limb movement disorder: Prevalence, clinical significance and treatment, Sleep Med. Rev., 10: 169-177, 2006
Garcia-Borreguero, D., Egatz, R., Winkelmann, J., Berger, K., Epidemiology of restless legs syndrome: The current status. Sleep Med. Rev., 10:153-167, 2006
Fox, G. N., Restless legs syndrome, American Family Physician, 33(1): 147-152, January 1986.
O'Keefe, S. T., Restless legs syndrome, a review. Arch. Internal Med., 165(3):243-248, 1996
Joy, M. S., Clonazepam: benzodiazepine therapy for the restless legs syndrome. ANNA J, 24(6):686-689, 1997
Trenkwaider et al., Periodic limb movements and restless legs syndrome, Neurol. Clinic, 14(3):629-650, 1996
Wetter, et al., Restless legs and periodic leg movements in sleep syndromes. J. Neurol., 244(4 Suppl. 1):S37-45, 1999
Hornyak et al., Magnesium therapy for periodic leg movements related insomnia and RLS: an open. pilot study, Sleep, 21(5):501-505, 1998
Saletu et al, Sleep laboratory studies in restless legs syndrome patients as compared with normals and acute effects of ropinirole. 2. Findings on periodic leg movements, arousals and respiratory variables Neuropsychobiology. 2000;41 (4):190-9
Gorman, J Chromatogr B Biomed Sci Appl. 1999
Allen R P, Earley C J. Augmentation of the restless legs syndrome with carbidopa/levodopa. Sleep 1996;19(3):205-13
Silber, M. H., Restless Legs Syndrome, Mayo Clin Proc. 72 (3):261-264
Saletu et al., Restless legs syndrome (RLS) and periodic limb movement disorder (PLMD): acute placebo-controlled sleep laboratory studies with clonazepam, Eur. Neuropsychopoharmacol., 11(2):153-161, 2001
Saletu et al., EEG mapping in patients with restless legs syndrome as compared with normal controls Psychiatry Res. 2002 Aug. 20;115(1-2):49-61
Saletu et al, Sleep Medicine Volume 3, Supplement 1, November 2002, Pages S35-S42
Silber, M. H., Restless Legs Syndrome, Mayo Clin Proc 72:261-264, 1997
Trenkwalder, C., The weight of evidence for ropinirole in restless leg syndrome, Eur J Neurol.13 Suppl 3:21-30, 2006
FDA approved labeling, 2004 for NDA 20-717/S-005 &S-008
Ropinirole US package insert
ICSD definition 780.524
ICSD definition 780.52-5
ICSD definition 780.53-1

1-11. (canceled)

12-14. (canceled)

15. A method of diminishing undesired involuntary limb movements of restless leg syndrome in an adult human, comprising the step of:
administering to an adult human having restless leg syndrome a pharmaceutically effective amount of a compound selected from the group consisting of modafinil, a pharmaceutically acceptable salt of modafinil, an enantiomer of modafinil or a pharmaceutically acceptable salt of an enantiomer of modafinil.

16. The method of claim 15, wherein said administering step comprises administering one of any of the enantiomers of modafinil.

17. The method of claim 15, wherein said administering step comprises administering one of any of the polymorphic forms of modafinil.

18. The method of claim 15, wherein the administering step comprises administering a dose of 10-100 mg of the compound.

19. The method of claim 18, wherein the dose is in the range of 25-100 mg of the compound.

20. The method of claim 15, wherein the administering step comprises continuous daily administration of the compound.

21. The method of claim 20, wherein the administering step comprises administering a daily dose of 10-100 mg of the compound.

22. The method of claim 21, wherein the daily dose is in the range of 25-100 mg of the compound.

23. The method of claim 1, wherein the administering step is performed in the evening pre-retiring to sleep.

24. The method of claim 20, wherein the administering step is performed in the evening pre-retiring to sleep.
